(19) United States
(12) Patent Application Publication
(10) Pub. No.: US 2024/0156646 A1
(43) Pub. Date: May 16, 2024

(54) NEGATIVE PRESSURE DRAINAGE SYSTEM

(71) Applicant: SHENZHEN BAO'AN PEOPLE'S HOSPITAL, SHENZHEN (CN)

(72) Inventors: XIAOHUA PAN, SHENZHEN (CN); XIAOMIN WU, SHENZHEN (CN); BIN BIAN, SHENZHEN (CN); ZHUOHENG PAN, SHENZHEN (CN); WEIBIN SU, SHENZHEN (CN)

(21) Appl. No.: 18/507,340

(22) Filed: Nov. 13, 2023

(30) Foreign Application Priority Data
Nov. 14, 2022 (CN) 202223032996.3

(51) Int. Cl. A61F 13/00; A61F 13/42; A61M 1/00

(52) U.S. Cl. CPC A61F 13/00068; A61F 13/42; A61M 1/96; A61M 1/966

(57) ABSTRACT

A negative pressure drainage system is disclosed, including a dressing for being applied to a wound surface of a patient; a collecting device, buried in the dressing for collecting a pressure at the dressing in real time to obtain pressure data; a receiving device, wirelessly connected with the collecting device, where the collecting device wirelessly is configured for transmitting the pressure data to the receiving device. Enabling the negative pressure drainage system to satisfy the above structure is beneficial to collecting the pressure data at the dressing in real time, thereby realizing real-time monitoring of a negative pressure at the wound surface.

### CROSS-REFERENCE TO RELATED APPLICATION

This application is based on and claims the benefit of priority from Chinese Patent Application No. 202223032996.3, filed on 14 Nov. 2022, the entirety of which is incorporated by reference herein.

### TECHNICAL FIELD

The present disclosure relates to the technical field of negative pressure drainage, and in particular to a negative pressure drainage system.

### BACKGROUND

Negative pressure drainage technology involves wrapping the patient's wound surface with a dressing, providing a negative pressure environment to the dressing site through a negative pressure device so as to create a tractive force on the dressing at the wound surface, and discharging necrotic tissues and exudate from the wound surface through drainage, thus improving the wound surface environment and promoting the healing of the wound surface.

The negative pressure value at the wound surface is a dynamic value, which is influenced by structures of the components in the negative pressure device. Different negative pressure environments are needed for healing in different healing processes. The current negative pressure drainage system generally cannot monitor the negative pressure at the wound surface in real time, and doctors need to judge the healing situation subjectively by observing the wound surface during a process of changing the dressing, which is complicated and may easily disrupt the normal healing process of the wound surface.

### SUMMARY

An objective of the present disclosure is to provide a negative pressure drainage system, which can solve the problem that a negative pressure at a wound surface cannot be monitored in real time.

In order to achieve the objective of the present disclosure, the present disclosure provides the following technical solution.

The present disclosure provides a negative pressure drainage system, comprising a dressing for being applied to a wound surface of a patient; a collecting device, buried in the dressing for collecting a pressure at the dressing in real time to obtain pressure data; a receiving device, wirelessly connected with the collecting device, wherein the collecting device wirelessly is configured for transmitting the pressure data to the receiving device.

In an embodiment, the collecting device comprises a first wireless module, the receiving device comprises a second wireless module, the first wireless module is wirelessly connected with the second wireless module for realizing wireless communication between the collecting device and the receiving device.

In an embodiment, the negative pressure drainage system further comprises a terminal, the receiving device is wirelessly connected with the terminal, the receiving device wirelessly transmits the pressure data to the terminal, and the terminal is configured for processing the pressure data.

In an embodiment, the negative pressure drainage system further comprises a negative pressure device, the negative pressure device is at least partially buried in the dressing, the negative pressure device is configured for providing a negative pressure at the dressing, the negative pressure device is electrically connected with the terminal, and the terminal is configured for adjusting a pressure provided by the negative pressure device according to the pressure data.

In an embodiment, the negative pressure device comprises a conduction pipe, the conduction pipe extends into the dressing, the conduction pipe is configured for conveying waste liquid at the dressing, and the collecting device is configured for collecting a pressure of the conduction pipe in the dressing.

In an embodiment, the terminal is configured for adjusting the negative pressure device to enable the pressure data to be within a preset pressure range.

In an embodiment, the negative pressure drainage system further comprises an alarm apparatus, the alarm apparatus is electrically connected with the terminal, and the terminal controls the alarm apparatus to give an alarm when a time duration for which the pressure data deviates from the preset pressure range exceeds a preset alarm time.

In an embodiment, the receiving device further comprises a display, and the display is configured for displaying the pressure data in real time.

In an embodiment, the negative pressure drainage system comprises a plurality of collecting devices and a plurality of receiving devices, the collecting devices correspond to the receiving devices one by one, the terminal is electrically connected with the plurality of receiving devices, and the display is further configured for displaying attribute information of each of the receiving devices.

In an embodiment, the receiving device further comprises a storage, the storage is configured for storing the pressure data, the storage is electrically connected with the terminal, and the terminal is configured for reading the pressure data of the storage.

In the negative pressure drainage system provided by the present disclosure, the dressing is applied to the wound surface of a patient, and the dressing provides a negative pressure for the wound surface to promote the healing of the wound surface. The collecting device is buried in the dressing, and the collecting device is configured for collecting a pressure at the dressing in real time to obtain pressure data, thus realizing real-time collection of the pressure data at the wound surface. The collecting device transmits the pressure data collected in real time to the receiving device, which realizes the collection of the pressure data at the wound surface. By reading the pressure data in the receiving device, the negative pressure at the wound surface can be analyzed and monitored.

### BRIEF DESCRIPTION OF THE DRAWINGS

In order to more clearly explain the technical solutions in the embodiments of the present disclosure or in the prior art, the accompanying drawings required in the description of the embodiments or the prior art will be briefly introduced below. Obviously, the accompanying drawings in the following description are only some embodiments of the present disclosure. For those of ordinary skills in the art, other accompanying drawings may be obtained according to these accompanying drawings without creative work.

FIG. 1 is a systematic schematic diagram of a negative pressure drainage system according to an embodiment of the present disclosure;

FIG. 2 is a schematic diagram of pressure data transmission of a negative pressure drainage system according to an embodiment of the present disclosure; and

FIG. 3 is a structural schematic diagram of a negative pressure drainage system according to an embodiment of the present disclosure.

### REFERENCE NUMERALS

10 refers to collecting device, 20 refers to receiving device, 30 refers to negative pressure device, refers to dressing, 50 refers to terminal, 60 refers to alarm apparatus;
11 refers to first wireless module;
21 refers to second wireless module, 22 refers to display, 23 refers to storage; and
31 refers to conduction pipe, and 32 refers to control valve.

### DETAILED DESCRIPTION

The technical solutions in the embodiments of the present disclosure will be described clearly and completely in conjunction with the accompanying drawings in the embodiments of the present disclosure. Obviously, the described embodiments are only a part of the embodiments of the present disclosure, but not all the embodiments. Based on the embodiments in the present disclosure, all other embodiments obtained by those of ordinary skills in the art without creative work shall fall within the protection scope of the present disclosure.

It should be noted that when a component is referred to as being “fixed” to another component, the component may be directly fixed to another component, or there may exist an intermediate therebetween. When a component is considered to be “connected” with another component, the component may be directly connected with another component, or there may be exist an intermediate therebetween.

Unless otherwise defined, all technical and scientific terms used in the present disclosure have the same meaning as commonly understood by those of ordinary skills in the art. The terms used in the description of the present disclosure is only for the purpose of describing specific embodiments and is not intended to limit the present disclosure. The term “and/or” as used in the present disclosure comprises any and all combinations of one or more related listed items.

Some embodiments of the present disclosure will be described in detail in conjunction with the accompanying drawings. In the case of no conflict, the following embodiments and features in the embodiments may be combined with each other.

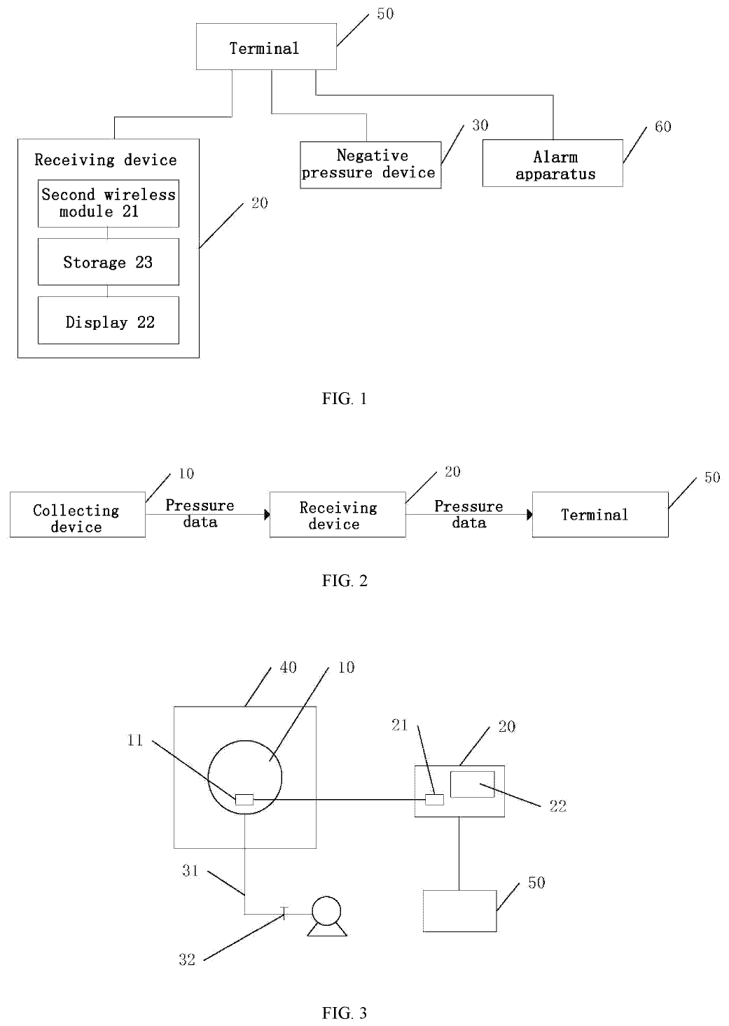

Embodiments of the present disclosure provide a negative pressure drainage system, referring to FIG. 1, FIG. 2 and FIG. 3, the negative pressure drainage system comprises a dressing 40, a collecting device 10 and a receiving device 20, wherein the dressing 40 is configured for being applied to a wound surface of a patient and providing a negative pressure at the wound surface to promote healing of the wound surface; the collecting device 10 is buried in the dressing 40, the collecting device 10 is configured for collecting a pressure at the dressing 40 in real time, and obtaining pressure data; and the receiving device 20 is wirelessly connected with the collecting device 10, and the receiving device is configured for receiving the pressure data in real time.

The dressing 40 may adopt a porous sponge dressing 40, or adopt a composite dressing 40 consisting of a porous sponge dressing 40 and an isolation pad. The isolation pad dressing 40 generally adopts Vaseline gauze, and may also be a porous isolation pad made of a hydrophilic and water-absorbent gel membrane by perforation. The isolation pad has good biocompatibility and hydrophilic surface, and is soft, smooth and nano-porous, which can be used as a drug carrier.

Different negative pressure environments need to be provided at different healing stages of the wound surface according to the situation, so as to achieve a good healing effect. However, the hardness of the dressing 40 will increase after long-term use, and the deformability of the dressing 40 will decrease, which will affect the negative pressure provided by the dressing 40 for the wound surface, leading to instability in the negative pressure environment at the wound surface. Burying the collecting device 10 in the dressing 40 to collect the pressure at the dressing 40 in real time is beneficial to obtaining a real-time pressure data at the dressing 40.

Optionally, the collecting device 10 comprises a pressure sensor, a probe of the pressure sensor is waterproof, and the pressure sensor is configured for collecting the pressure at the dressing 40 in real time.

The receiving device 20 is wirelessly connected with the collecting device 10, that is, only the collecting device 10 is arranged at the dressing 40 and is connected with the dressing 40, and the receiving device 20 is arranged outside the dressing 40, which is beneficial to reducing the number of devices connected with the dressing 40, simplifying the arrangement at the dressing 40 and improving the portability of the collecting device 10. Meanwhile, the collecting device 10 transmits the pressure data to the receiving device 20 through the wireless transmission. The transmission of the pressure data to the receiving device 20 is also realized. Compared with wired connection between the collecting device 10 and the receiving device 20, the data transmission mode is more convenient and is more conducive to activities of a patient.

The collecting device 10 transmits the collected pressure data to the receiving device 20 in real time, and the real-time monitoring of the negative pressure at the wound surface can be realized by reading the pressure data at the receiving device 20. This avoids the need to remove the dressing 40 to collect pressure data or to monitor the condition of the wound surface, which could otherwise interfere with the healing process of the wound surface. This facilitates the collection of pressure data to be more convenient.

Optionally, the receiving device 20 comprises a data interface, the data interface is configured for transmitting the pressure data to realize reading and copying of the pressure data. Analyzing the obtained pressure data can realize the real-time monitoring of the negative pressure at the wound surface.

The dressing 40 is applied at the wound surface of a patient, and provides the negative pressure for the wound surface, the collecting device 10 is buried in the dressing 40 and is configured for collecting the pressure at the dressing 40 in real time to obtain the pressure data, thus realizing the real-time collection of the pressure data at the wound surface. The collecting device 10 transmits the pressure data collected in real time to the receiving device 20, thus realizing the collection of the pressure data at the wound surface. The negative pressure at the wound surface may be analyzed and monitored by reading the pressure data in the receiving device 20. Meanwhile, the receiving device 20 is wirelessly connected with the collecting device 10, and the pressure data is transmitted in a wireless manner, which further improves the portability of the collecting device 10 and is more conducive to the activities of the patient.

In an embodiment, referring to FIG. 1, FIG. 2 and FIG. 3, the collecting device 10 comprises a first wireless module 11, the receiving device 20 comprises a second wireless module 21, and the first wireless module 11 is wirelessly connected with the second wireless module 21 for realizing wireless communication between the collecting device 10 and the receiving device 20.

Optionally, the collecting device 10 comprises a pressure sensor and a first wireless module 11, the pressure sensor is electrically connected with the first wireless module 11. The receiving device comprises a storage 23 and a second wireless module 21, the storage 23 is electrically connected with the second wireless module 21. The pressure sensor transmits the pressure data collected in real time to the second wireless module 21 through the first wireless module 11, after the second wireless module 21 receives the pressure data, the pressure data is stored in the storage 23, and the pressure data may be analyzed by reading the pressure data in the storage 23, so that the real-time monitoring of the negative pressure at the wound surface may be realized.

The collecting device 10 comprises the first wireless module 11, the receiving device 20 comprises the second wireless module 21, the first wireless module 11 is wirelessly connected with the second wireless module 21 for realizing the wireless communication between the collecting device 10 and the receiving device 20, which is beneficial to realizing the wireless transmission of the pressure data, thereby simplifying the connection between the collecting device 10 and the receiving device, and improving the portability of the collecting device 10. Compared with the wired connection between the collecting device 10 and the receiving device 20, the data transmission mode is more convenient, which is more beneficial to the activities of the patient.

In an embodiment, referring to FIG. 1, FIG. 2 and FIG. 3, the negative pressure drainage system further comprises a terminal 50, the receiving device 20 is wirelessly connected with the terminal 50, the receiving device 20 wirelessly transmits the pressure data to the terminal 50, and the terminal 50 is configured for processing the pressure data.

Optionally, the receiving device 20 comprises a data interface, the terminal 50 comprises a data connector, the data interface is connected with the data connector, so that the receiving device 20 is electrically connected with the terminal 50, and the pressure data at the receiving device 20 is transmitted to the terminal 50.

Optionally, the terminal 50 comprises a third wireless module, and the second wireless module 21 is wirelessly connected with the third wireless module for realizing wireless communication between the receiving device 20 and the terminal 50. To be specific, the collecting device 10 transmits the pressure data collected in real time to the second wireless module 21 through the first wireless module 11, and after the second wireless module 21 receives the pressure data, the pressure data received in real time is transmitted to the third wireless module through the second wireless module 21. After the terminal 50 receives the real-time pressure data through the third wireless module, the pressure data is analyzed at the terminal 50, so that the real-time monitoring of the negative pressure at the wound surface can be realized.

Optionally, the terminal 50 is provided with a displayer, the displayer is configured for displaying the pressure data, which is convenient for a doctor to observe and analyze the negative pressure of the wound surface according to the pressure data to obtain an analysis result. The terminal 50 is further provided with a memory, the memory is configured for storing the pressure data and the analysis result of the pressure data to realize the storage and backup of the pressure data and the analysis result. The terminal 50 may further be connected with a cloud and upload the pressure data and the analysis result to the cloud for other relevant people to consult. The terminal 50 is provided with a program for automatically analyzing the pressure data. After receiving the pressure data, the program automatically analyzes the pressure data and obtains a preliminary analysis result for doctor's reference.

The terminal 50 comprises but not limited to a computer, a mobile phone, a tablet personal computer and other devices.

The negative pressure drainage system further comprises the terminal 50, the receiving device is wirelessly connected with the terminal 50, the receiving device 20 wirelessly transmits the pressure data to the terminal 50, and the terminal 50 is configured for processing the pressure data, which is beneficial to realizing the wireless transmission of the pressure data between the receiving device 20 and the terminal 50, thereby improving the portability of the receiving device 20. Meanwhile, the terminal 50 processes the pressure data, which is beneficial for the pressure data collected in real time by the collecting device 10 to be transmitted to the terminal 50 in real time after being received by the receiving device 20, so that the terminal 50 can read and analyze the pressure data in real time, thereby realizing the real-time monitoring and analysis of the negative pressure at the wound surface.

In an embodiment, referring to FIG. 1 and FIG. 3, the negative pressure drainage system further comprises a negative pressure device 30, the negative pressure device 30 is at least partially buried in the dressing 40, and the negative pressure device 30 is configured for providing negative pressure at the dressing 40.

Optionally, the negative pressure device 30 comprises a conduction pipe 31, a waste liquid bottle, an air guide pipe and a vacuum pump. One end of the conduction pipe 31 is communicated with the dressing 40, and the other end is communicated with the waste liquid bottle, the waste liquid bottle is configured for collecting waste liquid at the dressing 40, and the conduction pipe 31 is configured for conveying the waste liquid such as necrotic tissues and exudate at the dressing 40 into the waste liquid bottle to be collected. One end of the air guide pipe is communicated with the waste liquid bottle, and the other end is communicated with the vacuum pump, the air guide pipe is configured for exhausting the air in the waste liquid bottle, and a volume of the air in the waste liquid bottle exhausted by the air guide pipe is the same as a volume of the waste liquid conveyed into the waste liquid bottle by the conduction pipe 31, so as to ensure an air pressure balance in the waste liquid bottle and maintain normal operation of the negative pressure device 30. The vacuum pump is configured for providing the negative pressure, and the negative pressure provided by the vacuum pump enables the negative pressure device 30 to provide the negative pressure at the dressing 40, and enables the dressing 40 to generate a tractive force on the wound surface, so as to discharge the waste liquid at the wound surface through drainage, thereby improving the wound surface environment and promoting the healing of the wound surface.

When the tractive force provided by the negative pressure at the wound surface is too large, a large number of waste liquid will be sucked out, which will affect blood circulation at the wound surface, thereby hindering the healing process. When the tractive force provided by the negative pressure at the wound surface is too small, the drainage may fail, which leads to the accumulation of the waste liquid at the wound surface, similarly hindering the healing process.

The negative pressure device 30 is electrically connected with the terminal 50, the terminal 50 is configured for adjusting the pressure provided by the negative pressure device 30 according to the pressure data. Optionally, the negative pressure device 30 comprises a control valve 32, the control valve 32 is arranged between the vacuum pump and the air gas pipe. The negative pressure provided by the vacuum pump for the whole negative pressure device 30 may be adjusted by controlling an opening size of the control valve 32, the opening size of the control valve 32 may be controlled by the terminal 50 or adjusted manually.

Therefore, after the pressure at the dressing 40 is collected in real time by the collecting device and transmitted by the receiving device 20, the pressure data finally reaches the terminal 50, and the terminal 50 analyzes the pressure environment at the wound surface according to the pressure data collected in real time, and maintains the stability of the pressure data at the dressing 40 by adjusting the negative pressure device 30 when necessary, that is, maintains the stability of the negative pressure environment of the wound surface, thereby promoting the healing of the wound surface to avoid the impact on the healing process of the wound surface due to reasons such as aging of the dressing 40. Meanwhile, the terminal 50 may also adjust the pressure provided by the negative pressure device 30 according to different healing conditions, so that the pressure environment at the wound surface is matched with the healing process of the wound surface. Optionally, the healing process of the wound surface may be judged by the detection of the waste liquid in the waste liquid bottle.

The negative pressure drainage system further comprises a negative pressure device 30, the negative pressure device 30 is at least partially buried in the dressing 40. The negative pressure device is configured for providing a negative pressure for the dressing 40. The negative pressure device is electrically connected with the terminal 50, and the terminal 50 is configured for adjusting the pressure provided by the negative pressure device 30 according to the pressure data, which is beneficial to the real-time adjustment of the negative pressure environment of the wound surface by the terminal 50 to maintain the stability of the negative pressure environment of the wound surface, or to adjust the pressure provided by the negative pressure device 30 according to different healing processes of the wound surface, so that the pressure environment at the wound surface is matched with the healing process of the wound surface, thereby improving the wound surface environment and promoting the healing of the wound surface.

In on embodiment, referring to FIG. 1 and FIG. 3, the negative pressure device 30 comprises a conduction pipe 31, one end of the conduction pipe 31 extends into the dressing 40, so that the conduction pipe 31 is communicated with the dressing 40. The conduction pipe 31 is configured for conveying the waste liquid at the dressing 40, and the collecting device 10 is arranged in an area near one end of the conduction pipe 31 inserted into the dressing 40 for collecting a pressure of the conduction pipe 31 in the dressing 40.

Optionally, the negative pressure device 30 comprises a plurality of conduction pipes 31, the plurality of conduction pipes 31 are communicated with the same dressing 40, and the plurality of conduction pipes 31 are arranged on the dressing 40 at intervals, and the plurality of conduction pipes 31 convey the waste liquid at the dressing 40 at the same time, which is beneficial to enabling the negative pressure environment at the wound surface to be more balanced, and the collecting device 10 is configured for detecting a pressure of the plurality of conduction pipes 31 in the dressing 40, which is beneficial to improving the accuracy of the pressure detection at the dressing 40.

The negative pressure device 30 comprises the conduction pipe 31, the conduction pipe 31 extends into the dressing 40, the conduction pipe 31 is configured for conveying the waste liquid at the dressing 40, and the collecting device 10 is configured for collecting the pressure of the conduction pipe 31 in the dressing 40. Compared with the structure in which the collecting device 10 is arranged far away from the dressing 40, the pressure at the dressing 40 can be collected more accurately, which avoids the influence of other structures in the negative pressure drainage system on the pressure collection and improves the accuracy of the pressure collection at the dressing 40.

In an embodiment, referring to FIG. 1 and FIG. 3, the terminal 50 is provided with a pressure range, and the terminal 50 is configured for adjusting the negative pressure device 30 so that the pressure data can be within a preset pressure range.

The pressure range is a numerical range set by a doctor on the terminal 50 according to the healing situation of the wound surface, so as to keep the pressure at the wound surface within the pressure range to ensure the stability of the negative pressure environment of the wound surface, thereby ensuring a normal healing process of the wound surface. When the pressure data is collected by the collecting device 10 and the pressure data is transmitted to the receiving device 20, the terminal 50 reads the pressure data at the receiving device 20 again, and judges whether the real-time pressure data is too large or too small compared with the pressure range, then the negative pressure device 30 is adjusted correspondingly to reduce or increase the pressure provided by the negative pressure device 30 for the dressing 40, so as to keep the real-time pressure data collected by the collecting device 10 within the pressure range and maintain the dynamic stability of the pressure at the wound surface.

The terminal 50 is configured for adjusting the negative pressure device 30, so that the pressure data is set within the preset pressure range, which is beneficial for the terminal 50 to adjust the pressure at the dressing 40 through the negative pressure device 30 according to the preset pressure range, thus ensuring the stability of the negative pressure environment at the wound surface, maintaining the dynamic stability of the pressure at the dressing 40, and thereby ensuring the normal healing process of the wound surface.

In an embodiment, referring to FIG. 1, the negative pressure drainage system further comprises an alarm apparatus 60, the alarm apparatus 60 is electrically connected with the terminal 50, the terminal 50 is provided with an alarm time, and the terminal 50 controls the alarm apparatus 60 to give an alarm when a time duration for which the pressure data deviates from the preset pressure range exceeds a preset alarm time.

When the terminal 50 receives that the pressure data at the collecting device 10 deviates from the preset pressure range, the terminal 50 controls the negative pressure device 30 to adjust the pressure at the dressing 40. However, in the process of adjustment, the pressure data always deviates from the preset pressure range, and the terminal 50 judges that the pressure adjustment is out of control in the negative pressure drainage system, controls the alarm apparatus 60 to give an alarm when the time duration for which the pressure data deviates from the preset pressure range exceeds the preset alarm time. The negative pressure drainage system is manually checked after receiving an alarm signal.

Optionally, the terminal 50 is further provided with the pressure adjustment range for the negative pressure device 30. By adjusting the pressure at the dressing 40 within the pressure adjustment range, the negative pressure environment at the wound surface can be within a safe treatment range. If the pressure of the negative pressure device 30 is adjusted to an end value of the pressure adjustment range by the terminal 50, and the pressure data is still outside the pressure range, then the terminal 50 judges that the pressure adjustment in the negative pressure drainage system is out of control, and controls the alarm apparatus 60 to give an alarm.

The negative pressure drainage system further comprises an alarm apparatus 60, the alarm apparatus 60 is electrically connected with the terminal 50. The terminal 50 controls the alarm apparatus 60 to give an alarm when a time duration for which the pressure data deviates from the preset pressure range exceeds a preset alarm time, which is beneficial for the negative pressure drainage system to independently maintain the stability of the pressure at the dressing 40, and feedback in time when the pressure at the dressing 40 is unstable, thus ensuring the safety of the negative pressure drainage system during use.

In an embodiment, referring to FIG. 1 and FIG. 3, the receiving device 20 further comprises a display 22, the display 22 is configured for displaying pressure data in real time. After the collecting device 10 transmits the pressure data collected in real time to the receiving device 20, the receiving device 20 displays a pressure value at the dressing 40 in real time through the display 22.

The receiving device 20 further comprises a display 22, the display 22 is configured for displaying the pressure data in real time, which is beneficial to reading the pressure value at the dressing 40 in real time and realize the real-time monitoring of the negative pressure at the wound surface.

In an embodiment, referring to FIG. 1 and FIG. 3, the negative pressure drainage system comprises a plurality of collecting devices 10 and a plurality of receiving devices 20, the collecting device 10 corresponds to the receiving device 20 one by one, each dressing 40 is provided with one collecting device 10, and each collecting device 10 corresponds to one receiving device 20.

The terminal 50 is electrically connected with a plurality of receiving devices 20, each receiving device 20 has independent attribute information, such as ID and other equipment parameter information. The display 22 is configured for displaying the attribute information of each receiving device and the display 22 is also configured for displaying the pressure data in real time. Therefore, the dressing 40 corresponding to the pressure data displayed by the display 22 can be obtained by reading the attribute information displayed by the display 22, which is beneficial to distinguishing the pressure data at different dressings 40.

The terminal 50 may read the pressure data at different receiving devices 20 by selecting different attribute information, that is, reading the pressure data at different dressings 40, so as to monitor and analyze the pressure data at the designated dressings 40.

The negative pressure drainage system comprises a plurality of collecting devices 10 and a plurality of receiving devices 20, the collecting devices 10 correspond to the receiving devices 20 one by one, the terminal 50 is electrically connected with the plurality of receiving devices 20, the display 22 is configured for displaying the attribute information of each receiving device, which is beneficial for the terminal 50 to actively read the pressure data at the designated receiving device 20, monitor and analyze the pressure data at the designated dressing 40, and enable the processing of the pressure data by the terminal 50 to be more targeted.

In an embodiment, referring to FIG. 1, the receiving device 20 further comprises a storage 23, the storage 23 is configured for storing pressure data, the storage 23 is electrically connected with the terminal 50, and the terminal 50 is configured for reading the pressure data of the storage 23.

Optionally, the receiving device 20 further comprises a display 22, the display 22 is electrically connected with the storage 23, and the display 22 is configured for displaying pressure data at the storage 23.

The receiving device 20 further comprises the storage 23, the storage 23 is configured for storing pressure data, the storage 23 is electrically connected with the terminal 50, and the terminal 50 is configured for reading the pressure data of the storage 23, which is beneficial to storing the pressure data, collecting the pressure data in a treatment process, facilitating subsequent reading and analyzing of the pressure data.

In the description of the embodiments of the present disclosure, it should be noted that the orientation or position relationship indicated by the terms “center”, “upper”, “lower”, “left”, “right”, “vertical”, “horizontal”, “inside” and “outside” is based on the accompanying drawings, which is only for the convenience of describing the present disclosure and simplifying the description, rather than indicating or implying that the referred device or element must have a specific orientation, be constructed and operated in a specific orientation, and therefore should not be construed as a limitation to the present disclosure.

The above disclosure only illustrates the preferred embodiments of the present disclosure, and certainly, it may not be used for limiting the scope of the present disclosure. Those of ordinary skills in the art may understand all or part of the process of realizing the above embodiments, and the equivalent changes according to the claims of the present disclosure still fall within the scope of the present disclosure.

What is claimed is:

1. A negative pressure drainage system, comprising:
a dressing for being applied to a wound surface of a patient;
a collecting device, buried in the dressing for collecting a pressure at the dressing in real time to obtain pressure data; and
a receiving device, wirelessly connected with the collecting device, wherein the collecting device wirelessly is configured for transmitting the pressure data to the receiving device.

2. The negative pressure drainage system according to claim 1, wherein the collecting device comprises a first wireless module, the receiving device comprises a second wireless module, the first wireless module is wirelessly connected with the second wireless module for realizing wireless communication between the collecting device and the receiving device.

3. The negative pressure drainage system according to claim 1, wherein the negative pressure drainage system further comprises a terminal, the receiving device is wirelessly connected with the terminal, the receiving device wirelessly transmits the pressure data to the terminal, and the terminal is configured for processing the pressure data.

4. The negative pressure drainage system according to claim 3, wherein the negative pressure drainage system further comprises a negative pressure device, the negative pressure device is at least partially buried in the dressing, the negative pressure device is configured for providing a negative pressure at the dressing, the negative pressure device is electrically connected with the terminal, and the terminal is configured for adjusting a pressure provided by the negative pressure device according to the pressure data.

5. The negative pressure drainage system according to claim 4, wherein the negative pressure device comprises a conduction pipe, the conduction pipe extends into the dressing, the conduction pipe is configured for conveying waste liquid at the dressing, and the collecting device is configured for collecting a pressure of the conduction pipe in the dressing.

6. The negative pressure drainage system according to claim 4, wherein the terminal is configured for adjusting the negative pressure device to enable the pressure data to be within a preset pressure range.

7. The negative pressure drainage system according to claim 6, wherein the negative pressure drainage system further comprises an alarm apparatus, the alarm apparatus is electrically connected with the terminal, and the terminal controls the alarm apparatus to give an alarm when a time duration for which the pressure data deviates from the preset pressure range exceeds a preset alarm time.

8. The negative pressure drainage system according to claim 3, wherein the receiving device further comprises a display, and the display is configured for displaying the pressure data in real time.

9. The negative pressure drainage system according to claim 8, wherein the negative pressure drainage system comprises a plurality of collecting devices and a plurality of receiving devices, the collecting devices correspond to the receiving devices one by one, the terminal is electrically connected with the plurality of receiving devices, and the display is further configured for displaying attribute information of each of the receiving devices.

10. The negative pressure drainage system according to claim 3, wherein receiving device further comprises a storage, the storage is configured for storing the pressure data, the storage is electrically connected with the terminal, and the terminal is configured for reading the pressure data of the storage.
